(19) United States
(12) Patent Application Publication
(10) Pub. No.: US 2019/0192718 A1
(43) Pub. Date: Jun. 27, 2019

(54) DISINFECTANT DEVICE

(71) Applicant: JCS Biotech Ptd Ltd, Singapore (SG)

(72) Inventor: Jason Hock Huat Yeo, Singapore (SG)

(21) Appl. No.: 16/327,706

(22) PCT Filed: Aug. 23, 2017
(86) PCT No.: PCT/SG2017/050414

§ 371 Date: Feb. 22, 2019

(30) Foreign Application Priority Data
Aug. 23, 2016 (SG) 10201607023T

(51) Int. Cl. A61L 9/12

(52) U.S. Cl. CPC A61L 9/12; A61L 2209/135; A61L 2209/11

(57) ABSTRACT

A disinfection device comprising: a housing; a reservoir within said housing, said reservoir arranged to contain a liquid disinfectant; a vaporizer in fluid communication with the reservoir, said vaporizer arranged to vaporize the liquid mist; a nozzle in fluid communication with the vaporizer, said nozzle arranged to vent the vaporized disinfectant; a drive unit mounted to the housing for driving the disinfection device along a floor space; wherein the disinfection device is arranged to vaporize and diffuse disinfection mist whilst driving across said floor space.

### FIELD OF THE INVENTION

The invention relates to the disinfection of facilities such as healthcare facilities. In particular, the invention is directed to a device for maintaining a required level of disinfection.

### BACKGROUND

With the advent of super bugs and other antibiotic resistant organisms, maintaining a high level of disinfection is important to mitigate against cross infection between patients within healthcare facilities, for instance, retirement homes, hospital wards and emergency patient care.

It will be appreciated that a potential cause of cross contamination may occur when declining budgets, or employee neglect, leads to the omission of the disinfection of critical areas. This may consequently provide a foothold upon which potentially harmful organisms may thrive.

### SUMMARY OF INVENTION

In a first aspect, the invention provides a disinfection device comprising: a housing; a reservoir within said housing, said reservoir arranged to contain a liquid disinfectant; a vaporizer in fluid communication with the reservoir, said vaporizer arranged to vaporize the liquid mist; a nozzle in fluid communication with the vaporizer, said nozzle arranged to vent the vaporized disinfectant; a drive unit mounted to the housing for driving the disinfection device along a floor space; wherein the disinfection device is arranged to vaporize and diffuse disinfection mist whilst driving across said floor space.

Accordingly, the autonomous device according to the present invention provides a disinfecting mist according to the requirements of the facility in which the device is deployed. Specifically, by automating the disinfection process, neglect may be eliminated as a pre-disposing cause of such infection.

### BRIEF DESCRIPTION OF DRAWINGS

It will be convenient to further describe the present invention with respect to the accompanying drawings that illustrate possible arrangements of the invention. Other arrangements of the invention are possible and consequently, the particularity of the accompanying drawings is not to be understood as superseding the generality of the preceding description of the invention.

FIG. 1 is a cross sectional view of an autonomous disinfection device according to one embodiment of the present invention.

### DETAILED DESCRIPTION

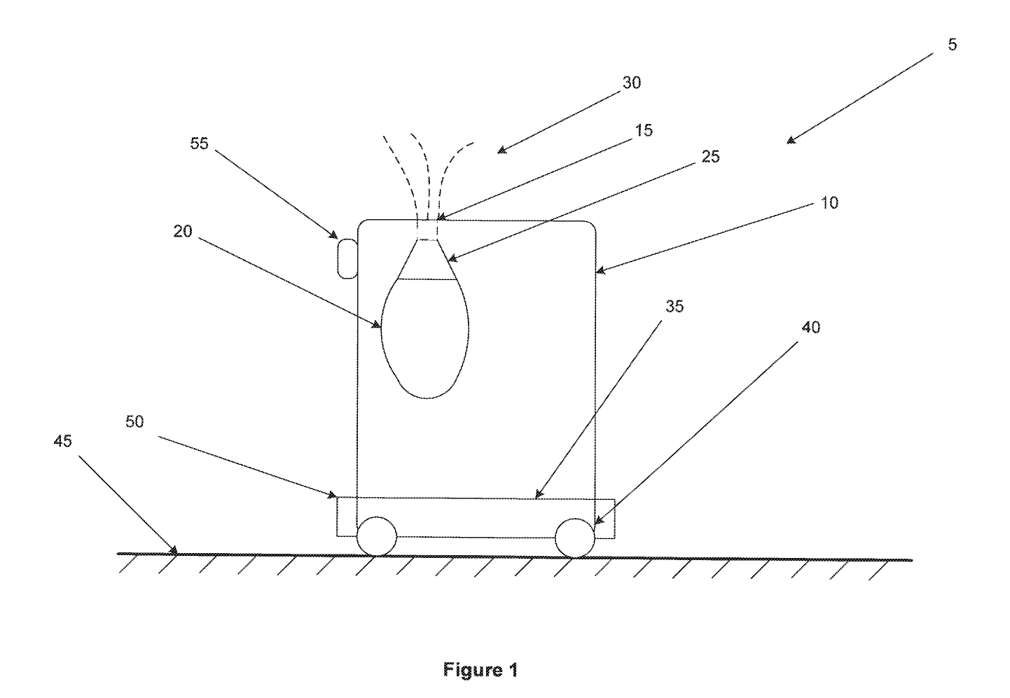

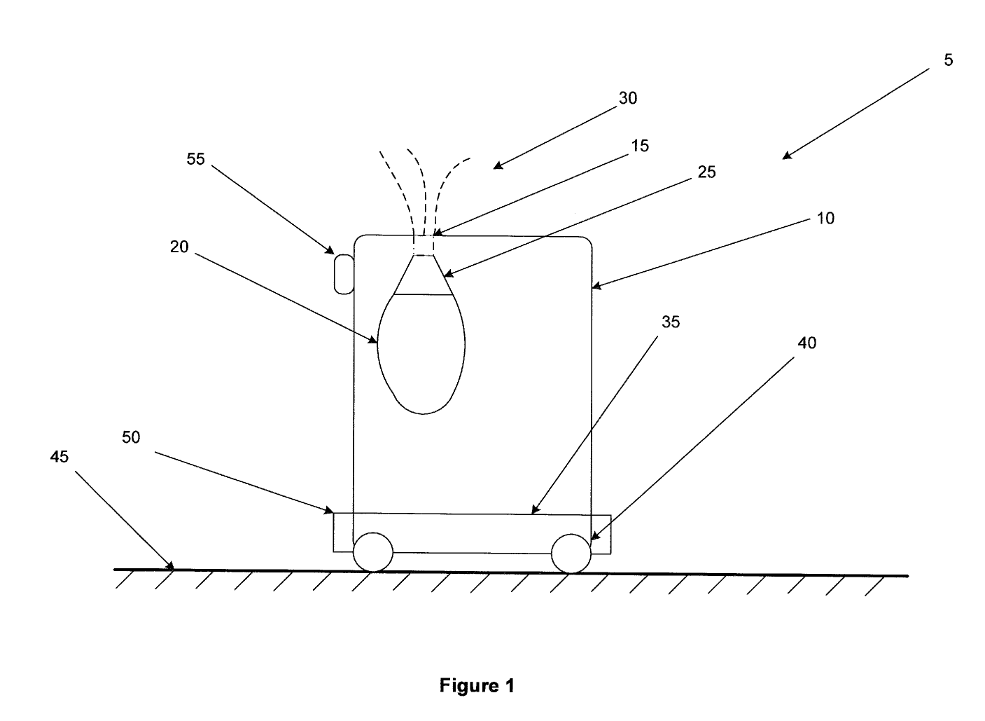

FIG. 1 is a cross sectional view of a disinfection device 5 according to one embodiment of the present invention. The disinfection device 5 includes a housing 10 arranged to move using a drive unit across a floor 45 in order to diffuse a disinfectant mist 30 from a vent 15 of the device 5.

Within the housing 10 is located a reservoir 20 of the disinfectant. Mounted to, or in fluid communication with, the reservoir 20 is a vaporizer 25 which may take the form of a transducer arranged to vaporize the disinfectant so as to distribute the vaporized disinfectant as a mist for diffusion in the open space.

In one embodiment the vaporizer may be an ultrasonic transducer applying a frequency in the range 17 kHz to 5 MHz. The mechanics by which the ultrasonic transducer atomizes the liquid form of mist is outside the present invention, but may include the application of a vibrating ultrasonic nozzle tip applied to the liquid in order to produce the atomized mist.

The drive unit includes a motor 35 and wheels 40 driven by said motor, as well as a steering assembly to turn the wheels in the desired direction. The motor 35 may be subject to a control system which provides directional control to the device, and in so doing, provide for an autonomous disinfection device, which is capable of controlling direction without intervention.

A form of directional control, according to one embodiment, may include a directional instruction whereby the autonomous device 5 operates around a floor space, then changing direction once an object is detected either by a proximity sensor or through a contact sensor 50 such as a bumper. On contacting an object the control system directs the device 5 to move in an alternative direction so as to randomly cover the floor space.

In an alternative embodiment the control system may have a predetermined path around the floor space. The predetermined path may be identified so as to direct the autonomous device 5 into areas which may not necessarily allow for easy diffusion of the mist. For instance, a predetermined path may be useful if the autonomous device 5 is required to enter several rooms, cubicles or a larger space separated by partitions which may interfere with free diffusion. By incorporating a predetermined path the control system may deliver the autonomous device 5 to various strategic and disparate location to ensure full distribution of the diffused mist.

In a still further embodiment, the autonomous device 5 may include a sensor 55 for tracking a path marked upon the floor space. Alternatively, the marked path on the floor space may be a contact strip arranged to contact the wheels 40. The contact strip may include a metal strip which indicates to the control system the path to be followed by the autonomous unit 5.

The device, however, may not be autonomous or semi-autonomous in that it may be directed by an operator such as through a remote control and thus undergo supervision to direct the device to the correct location. Under this remote control, the control may be fully directive such that the operator indicates the speed and direction by which the device 5 will travel. Alternatively, the remote control may be semi-autonomous in that in the absence of a direct intervention by an operator, the control system will adopt any of the abovementioned directional controls which may be overridden by an operator for a number of reasons including to move the device to a different location.

The generation of the mist may be a continuous generation until the reservoir is empty. In this case, the reservoir may continuously produce the mist until exhausted. This may, for instance, take an hour to finish the reservoir, depending upon the reservoir volume and rate of mist generation.

Alternatively, the mist generation may be intermittent with a controllable period in between generation periods. In a still further embodiment the generation of mist may be controllable by an operator so that the operator may deliver the autonomous device to a particular floor space to generate the mist then relocated to a different location during which the mist generation is halted.

1. A disinfection device comprising:
a housing;
a reservoir within said housing, said reservoir arranged to contain a liquid disinfectant;
a vaporizer in fluid communication with the reservoir, said vaporizer arranged to vaporize the liquid mist;
a nozzle in fluid communication with the vaporizer, said nozzle arranged to vent the vaporized disinfectant;
a drive unit mounted to the housing for driving the disinfection device along a floor space;
wherein the disinfection device is arranged to vaporize and diffuse disinfection mist whilst driving across said floor space.

2. The disinfection device according to claim 1, further including a control system in communication with the drive unit, such that the device is an autonomous device, wherein the control system is arranged to apply directional control to the drive unit to drive the device along a path.

3. The disinfection device according to claim 2, wherein the path is stored in the control system, the control system arranged to apply the directional control by instructing the drive unit to follow said path.

4. The disinfection device according to claim 2, wherein the path is stored in the control system.
